(19) United States
(12) Patent Application Publication
(10) Pub. No.: US 2022/0291529 A1
(43) Pub. Date: Sep. 15, 2022

(54) PROTECTIVE COVER FOR EYEWEAR

(71) Applicant: Fox Head, Inc., Irvine, CA (US)

(72) Inventors: David Andrew Munn, Mission Viejo, CA (US); Chad Michael McKonly, Irvine, CA (US); Raul Guillermo Reyes, Anaheim, CA (US)

(21) Appl. No.: 17/689,942

(22) Filed: Mar. 8, 2022

Related U.S. Application Data
(60) Provisional application No. 63/158,888, filed on Mar. 10, 2021.

(51) Int. Cl. G02C 13/00; B08B 1/00; B08B 1/02; B08B 17/04

(52) U.S. Cl. CPC G02C 13/006; B08B 1/005; B08B 1/02; B08B 17/04

(57) ABSTRACT

A protective cover for an eyewear including a lens cleaning system and a frame. The protective cover includes a first end portion, a second end portion, a lens opening configured to allow access to a lens of the eyewear, an outer side, and an inner side. The inner side includes a shape that corresponds to a shape of at least a first side of the frame of the eyewear and at least a portion of the lens cleaning system. The inner side may also include a first cleaning system receiver configured to cover a first portion of the lens cleaning system, and a second cleaning system receiver configured to cover a second portion of the lens cleaning system. The protective cover encloses a first side of the frame and the lens cleaning system. The protective cover reduces a failure of the lens cleaning system due to debris.

### CROSS-REFERENCE TO RELATED APPLICATION(S)

This non-provisional patent application claims the benefit of and priority to U.S. Provisional Patent Application No. 63/158,888, titled PROTECTIVE COVER FOR EYEWEAR, filed Mar. 10, 2021, which is incorporated herein in its entirety by reference thereto.

### TECHNICAL FIELD

The subject matter described herein relates generally to eyewear, and more specifically to a protective cover for eyewear.

### BACKGROUND

Eyewear, such as goggles, may be worn to protect a wearer's eyes from radiation and foreign objects. In active sports, particularly, the lens of the eyewear may become dirty or collect dust, snow or other type of debris that block the view of the wearer. This can be undesirable for the wearer when participating in active sports. Additionally, eyewear may include one or more systems, such as roll-off systems, for protecting or cleaning the lens of the eyewear. Roll-off systems may include disposable lenses, such as lens films, that are packaged in a canister. The lens films may be advanced across the lens of the eyewear. During use, such as during active sports, dirt, mud, water, and/or other debris may contact the roll-off systems or become attached to the roll-off systems.

### SUMMARY

According to some aspects of the present technology, an eyewear system includes an eyewear and a protective cover. The eyewear may include a frame, a lens disposed within the frame, a lens cleaning system for cleaning the lens, a first side extending across the frame, and a second side opposite the first side and configured to contact a face of a wearer of the eyewear when worn. The protective cover may include a first end portion, a second end portion, a lens opening configured to allow access to the lens, an outer side, and an inner side. The lens opening may be positioned laterally between the first end portion and the second end portion. The inner side may include a shape that corresponds to a shape of at least the first side of the frame and at least the portion of the lens cleaning system. The inner side may also include a first cleaning system receiver configured to cover a first portion of the lens cleaning system. The first cleaning system receiver may be positioned at the first end portion. The second cleaning system receiver may cover a second portion of the lens cleaning system. The second cleaning system receiver may be positioned at the second end portion. The protective cover may be coupled to the frame. The protective cover may enclose at least the first side and at least the portion of the lens cleaning system. The protective cover may reduce a failure of the lens cleaning system due to debris contacting the lens cleaning system.

In some aspects of the present technology, the lens cleaning system may include a roll-off system.

The first portion of the lens cleaning system may be a first portion of a first canister that is configured to store at least a first portion of a film for cleaning the lens of the eyewear. The second portion of the lens cleaning system may be a second portion of a second canister that is configured to store at least a second portion of the film.

The protective cover includes a scraper configured to tear a film of the lens cleaning system. The scraper may include a first scraper positioned on the first end portion. The scraper may also include a second scraper positioned on the second end portion. The first scraper and the second scraper may each define a rip-off interface configured to facilitate detachably coupling a rip-off film sheet across the lens. The scraper may include a plurality of raised blades.

In some aspects of the present technology, the protective cover further comprises a top portion and a bottom portion, opposite the top portion. The top portion may extend from the first end portion to the second end portion. The bottom portion may extend from the first end portion to the second end portion. The top portion, the bottom portion, the first end portion, and the second end portion may surround the lens opening of the cover.

In some aspects of the present technology, the protective cover includes a flexible material. In some aspects, the protective cover includes a silicone material.

The protective cover may be coupled to the frame of the eyewear via one or more of an adhesive, a snap-fit arrangement, and a mechanical fastener.

In some aspects, the eyewear comprises a goggle.

The foregoing summary is illustrative only and is not intended to be in any way limiting. In addition to the illustrative aspects, embodiments, and features described above, further aspects, implementations, and features will become apparent by reference to the drawings and the following detailed description.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a perspective view of an eyewear system including a protective cover and an eyewear, consistent with implementations of the current subject matter;

FIG. 2 depicts a front view of an eyewear system including a protective cover and an eyewear, consistent with implementations of the current subject matter;

FIG. 3 depicts an exploded view of an eyewear system including a protective cover and an eyewear, consistent with implementations of the current subject matter;

FIG. 4 depicts a front perspective view of a protective cover, consistent with implementations of the current subject matter;

FIG. 5 depicts a rear perspective view of a protective cover, consistent with implementations of the current subject matter;

FIG. 6 depicts a perspective view of an eyewear system including a protective cover and an eyewear, consistent with implementations of the current subject matter;

FIG. 7 depicts a side view of an eyewear system including a protective cover and an eyewear, consistent with implementations of the current subject matter;

FIG. 8 depicts a front view of an eyewear system including a protective cover and an eyewear, consistent with implementations of the current subject matter;

FIG. 9 depicts a top view of an eyewear system including a protective cover and an eyewear, consistent with implementations of the current subject matter;

FIG. 10 depicts a front view of an eyewear system including a protective cover and an eyewear, consistent with implementations of the current subject matter;

FIG. 11 depicts a front view of an eyewear system including a protective cover and an eyewear, consistent with implementations of the current subject matter;

FIG. 12 depicts a scraper of a protective cover, consistent with implementations of the current subject matter;

FIG. 13 depicts a tear-off post, consistent with implementations of the current subject matter;

FIG. 14 depicts a front view of an eyewear system including a protective cover and an eyewear, consistent with implementations of the current subject matter;

FIG. 15 depicts a front view of a protective cover, consistent with implementations of the current subject matter;

FIG. 16 depicts a front view of a protective cover, consistent with implementations of the current subject matter; and

FIG. 17 depicts a front view of a protective cover, consistent with implementations of the current subject matter.

### DETAILED DESCRIPTION

Various aspects of the disclosure will now be described with regard to certain examples and implementations, which are intended to illustrate but not to limit the disclosure. Nothing in this disclosure is intended to imply that any particular feature or characteristic of the disclosed implementations is essential. The scope of protection is defined by the claims that follow this description and not by any particular implementation described herein. Before turning to the figures, which illustrate example implementations in detail, it should be understood that the application is not limited to the details or methodology set forth in the description or illustrated in the figures. It should also be understood that the terminology is for the purpose of description only and should not be regarded as limiting.

Implementations herein generally relate to a protective cover for an eyewear, such as goggles, glasses, sunglasses, protective eyewear, and the like. As described in more detail herein, the protective cover may be coupled to the eyewear to protect at least a portion of the eyewear, such as a lens cleaning system, from debris, thereby preventing or reducing potential malfunction of the lens cleaning system and increasing the effectiveness of the eyewear.

The eyewear described herein separate from and/or coupled to the protective cover, may be used in a number of activities, including sports and athletics, such as extreme sports (e.g., motocross and snowmobiling); cycling activities, including auto racing, motorcycle riding and racing, BMX, mountain biking, etc.; with recreational vehicles including all-terrain vehicles (ATVs), dirt bikes, utility task vehicles (UTVs), snowmobiles, and other off-road vehicles; military applications; and/or construction applications. For example, in some implementations, the eyewear is a motocross goggle. In some implementations, the eyewear is a snowmobiling goggle, a snowboarding goggle, a mountain biking goggle, a motorcycle goggle, a sky diving goggle, or still another action or extreme sport goggle. In some implementations, the eyewear is or includes a swimming goggle or other sports goggle (e.g., used in basketball, baseball, etc.; Rec Specs®; etc.). Additionally and/or alternatively, the eyewear is or includes a visor coupled to a helmet (e.g., a football helmet, a motorcycle helmet, etc.). In some implementations, the eyewear is another type of eyewear used for construction, military applications, machining, carpentry, scientific experimentation, and/or the like. In some implementations, the eyewear is a traditional vision enhancing glasses (e.g., prescription glasses, etc.) and/or sunglasses.

In some implementations, the eyewear described herein includes a frame, a lens (e.g., a permanently attached or removable lens) disposed within the frame, and a lens cleaning system coupled to (e.g., releasably connected to or integrally formed with) the frame. The lens cleaning system may include a rip-off system and/or a roll-off system. The lens cleaning system may facilitate removing debris from a lens hindering the vision of the wearer of the eyewear when worn during the described activities. The lens cleaning system may be configured to facilitate selectively cleaning the lens in a relatively simple manner (e.g., easily, quickly, etc.), by removing debris that hinder the vision of the wearer from the lens, and the like. For example, the lens cleaning system may include a roll-off system. In the roll-off system, a roll of film that is selectively translated across the lens from one canister to another as the film becomes dirty (e.g., with dirt, mud, water, snow, and the like). The roll-off system may include film that is pulled, stretched, laid or otherwise applied across the lens of the eyewear, from one canister to another. The lens cleaning system may additionally and/or alternatively include a rip-off system having a plurality of rip-off film elements that couple to the eyewear and may be torn or ripped off one-by-one as each becomes dirty. The film may be made of transparent material, such as plastic. The material may be fully transparent, semi-transparent, tinted, or patterned or polarized in a manner that the film does not obstruct the wearer's view. The film may have a thickness and/or opacity that allows the wearer of the eyewear to see through the lens over which the film is positioned.

In particular, roll-off systems or other lens cleaning systems, may be mounted directly to the lens of eyewear, may be integrally formed with (e.g., molded with, form a part of, etc.) the frame, and/or may be detachable from at least a portion of the frame of the eyewear. The lens cleaning system may be coupled to the frame as outriggers and/or the outriggers may be separate coupled to the frame and/or the lens cleaning system. The lens cleaning systems may include a first canister integrally formed with and/or coupled to a first end portion of the frame and a second canister may be integrally formed with and/or coupled to a second lateral end of the frame. Other examples of eyewear with roll off systems include U.S. Patent Application Publication No. US 2021/0177659, titled Eyewear Frame with Integral Roll-off System, filed Dec. 13, 2019, and U.S. Patent Application Publication No. 2019/0142639, filed Oct. 23, 2018, each of which are incorporated herein in their entirety by reference thereto.

During the described activities, mud, dirt, liquids, or other debris may contact the eyewear and/or the lens cleaning system, hindering the vision of the wearer of the eyewear. In some situations, the debris may pack onto and/or become lodged in the eyewear, and in particular, the lens cleaning system. This may disrupt the lens cleaning system, making it more difficult for the lens cleaning system to translate and/or apply a new sheet when the currently applied sheet or portion of the film becomes dirty. In such circumstances, replenishing the film or sheet covering the lens of the eyewear may become difficult.

The protective cover consistent with implementations of the current subject matter may help to reduce, or prevent failure of the lens cleaning system of the eyewear when worn by the user. For example, the protective cover may include an inner side, an outer side, a first end portion, a second end portion, a lens opening positioned between the first and second end portions and configured to allow access to a lens of the eyewear. The inner side of the protective cover may include a shape that correspond to a shape of at least a first side of the frame of the eyewear that is configured to face away from the wearer when worn. The inner side may include a first cleaning system receiver and/or a second cleaning system receiver that covers and/or has a shape that corresponds to respective portions of the lens cleaning system of the eyewear. For example, in the case of a roll-off system, the first cleaning system receiver and the second cleaning system receiver may cover and/or have a shape that corresponds to respective canisters of the lens cleaning system. The shape, size, material, and/or the like of the protective cover allows the protective cover to couple to, enclose, and/or tightly seal around at least the first side (e.g., outwardly facing side) of the frame and lens cleaning system. Thus, the protective cover may reduce the surface area of the frame and/or the lens cleaning system that is exposed to mud, water, or other debris.

As noted above, the protective cover may tightly seal around, couple to, and/or enclose at least a portion of the eyewear, such as at least a portion of the frame and the lens cleaning system. In some implementations, the protective cover includes a rubber, silicone, and/or other flexible material that stretches over at least the first side of the frame and/or the lens cleaning system for a tight and/or seal, while allowing the lens cleaning system to function normally, without failure or with a reduced likelihood of failure due to contact with and buildup of the debris. Additionally and/or alternatively, as described herein the protective cover may be lined with adhesive or another coupling mechanism to further improve the fit and/or seal on the eyewear to reduce and/or prevent ingress of the debris into the interior of the protective cover where the debris may contact or become lodged onto the lens cleaning system of the eyewear.

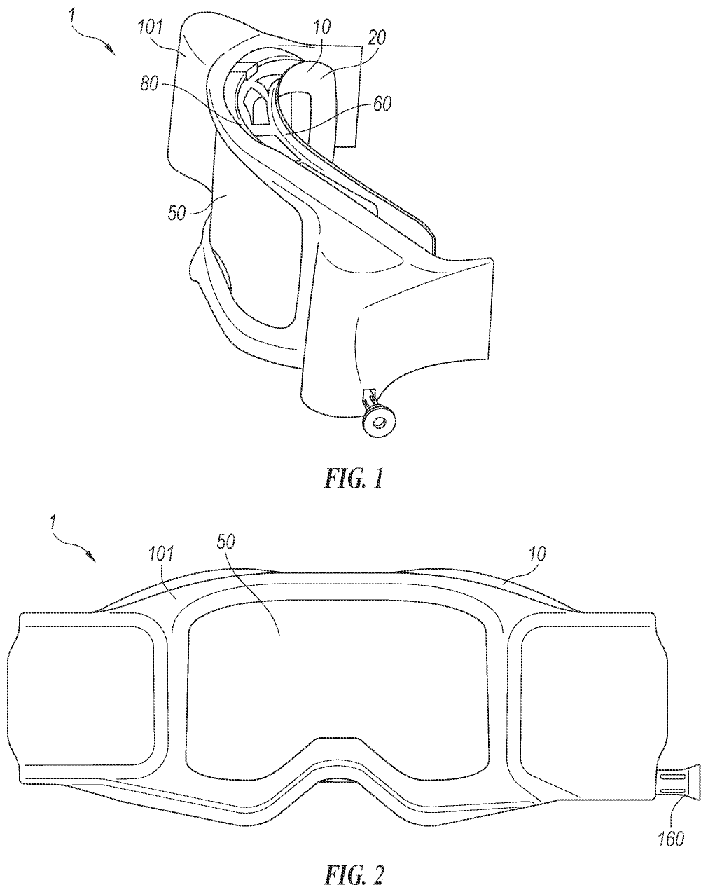

FIGS. 1-5 illustrate a perspective view of an example eyewear system 1, consistent with implementations of the current subject matter. The eyewear system 1 may include a protective cover 101 and an eyewear 10. In this example, the eyewear 10 is shown as a goggle 10. Though, as described herein, other types of eyewear may be implemented.

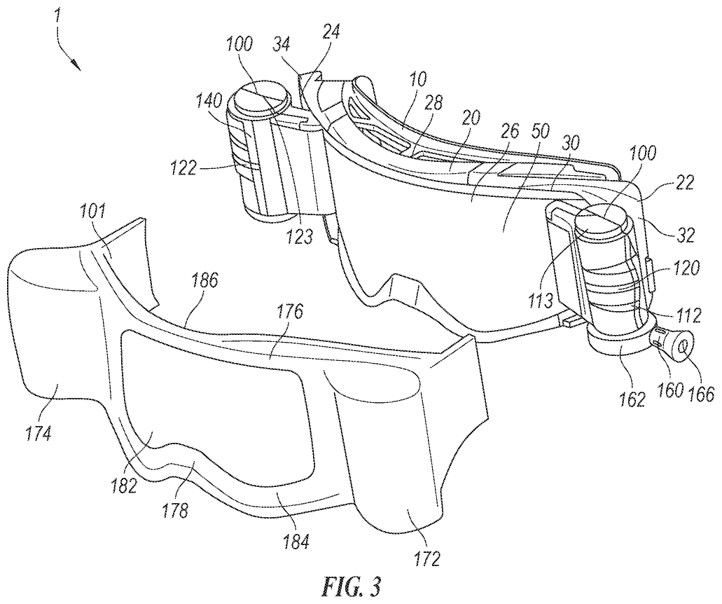

The goggle 10 includes a frame 20 having a frame body, a lens 50, a cushion member 60, such as a compressible member, an extender or bridge 80, and a lens cleaning system 100. In some implementations, the lens cleaning system 100 (see FIG. 3) may include a roll-off system and/or a rip-off system. The lens cleaning system 100 may be integrally formed (e.g., molded with, forms a part of, etc.) and/or coupled to the frame 20 and may facilitate selectively cleaning the lens 50. For example, the lens cleaning system 100 may remove debris from the lens 50 to provide an unobstructed view for the wearer in a relatively simple manner (e.g., easily, quickly, etc.), such as during a race, competition, and/or other activity. In some implementations, the lens cleaning system 100 is adhesively and/or mechanically secured to the frame 20. In some implementations, the lens cleaning system 100 may be attachable or detachable from the frame 20 in a pressure-fit or snap-fit manner.

Generally, the goggle 10 provides protection to the eyes and adjacent area of the face of a wearer. For example, the lens 50 and/or the frame 20 may intercept light, wind, rain, snow, water, particulate matter (e.g., dust, dirt, mud, etc.), and the like, to protect the eyes and/or various portions of the face of a wearer of the goggle 10. As shown in FIGS. 1-5, the frame 20 has a first end portion 22, an opposing second end portion 24, a first or front side, shown as exterior side 26, and an opposing second or rear side, shown as interior side 28.

The frame 20 may be shaped to correspond with anatomical features of a wearer's face (e.g., cheeks, nose, forehead, etc.) to facilitate a proper fit of the goggle 10 when worn. As shown in FIGS. 1-3, the compressible member 60 is coupled to the interior side 28 of the frame 20 by the bridge 80. For example, bridge 80 may space the compressible member 60 from the interior side 28 of the frame 20. In some implementations, the goggle 10 does not include the bridge 80 (e.g., the compressible member 60 is directly coupled to the interior side 28 of the frame 20, etc.). The compressible member 60 may thereby be positioned along the interior side 28 of the frame 20 such that the compressible member 60 is in contact with a wearer's face when the goggle 10 is worn. The bridge 80 and/or the compressible member 60 may include an impact attenuating material and/or cushion material (e.g., expanded polyurethane (EPU) foam, expanded polypropylene (EPP) foam, expanded polyethylene (EPE) foam, polyolefin foam, rubber, a flexible polymer material, etc.) to facilitate a snug and comfortable fit of the goggle 10 onto a wearer's face and/or to mitigate impact forces encountered by the goggle 10 (e.g., from flying debris, etc.).

As shown in FIGS. 1-3, the frame 20 defines a cavity or opening, shown as lens cavity 30. The lens cavity 30 may receive the lens 50 such that the lens 50 is disposed across and/or within, and supported by the frame 20.

As shown in FIGS. 1-3, the lens 50 includes a unitary, arcuate lens that extends across the entirety of the lens cavity 30 of the frame 20 (e.g., over both the left and right eyes of the wearer, from the first end portion 22 to the second end portion 24, etc.). In some implementations, the lens 50 includes dual lenses—one positioned over each of the left and right eyes of the wearer. In some implementations, the lens 50 is clear (e.g., substantially transparent, etc.). In some implementations, the lens 50 includes a tinted and/or polarized coating to shade a wearer's eyes from sunlight, reduce glare, improve contrast, and/or enhance depth perception. The tinted and/or polarized coating may be various colors (e.g., black, yellow, blue, green, brown, gray, red, etc.). In some implementations, the lens 50 includes a reflective coating (e.g., to prevent others from seeing where the wearer is looking, etc.). In some implementations, the lens 50 is a prescription lens configured to enhance the vision of a wearer of the goggle 10. In some implementations, the lens 50 is or includes a toroidal lens and/or a spherical lens (e.g., to prevent distortion in the vision of the wearer, etc.). In some implementations, the lens 50 is or includes a cylindrical lens. In other implementations, the lens 50 is still otherwise shaped.

As shown in FIG. 3, the first end portion 22 of the frame 20 defines a first interface (e.g., a slot, an aperture, a recess, a receiver, etc.), shown as first band interface 32, and the second end portion 24 of the frame 20 defines a second interface (e.g., a slot, an aperture, a recess, a receiver, etc.), shown as second band interface 34. The first band interface 32 and the second band interface 34 facilitate releasably coupling a head band to the frame 20 of the goggle 10. For example, the head band may include a first band end and a second band end. The first band end may have a first coupler (e.g., a connector, a clip, etc.) that is received by the first band interface 32 to releasably secure the first band end of the head band to the first end portion 22 of the frame 20. The second end may have a second coupler (e.g., a connector, a clip, etc.) that is received by the second band interface 34 to releasably secure the second end of the head band to the second end portion 24 of the frame 20. In other implementations, the head band is fixed to the frame 20. In some implementations, the head band includes an elastic band and/or has a length adjuster element configured to facilitate selectively adjusting (e.g., extending, shortening, etc.) the size of the head band. The head band may thereby allow the goggle 10 to fit various sized heads.

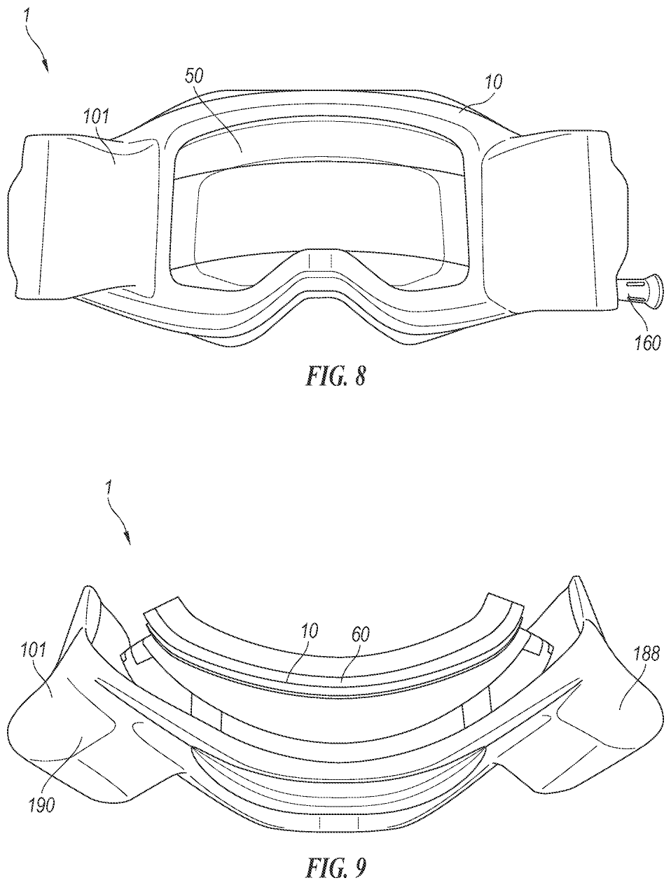

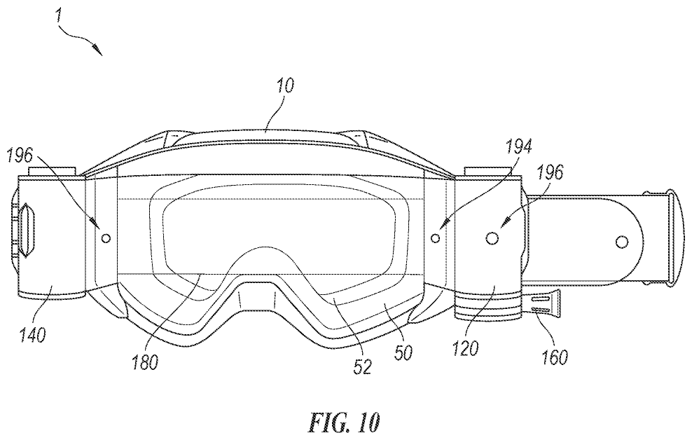

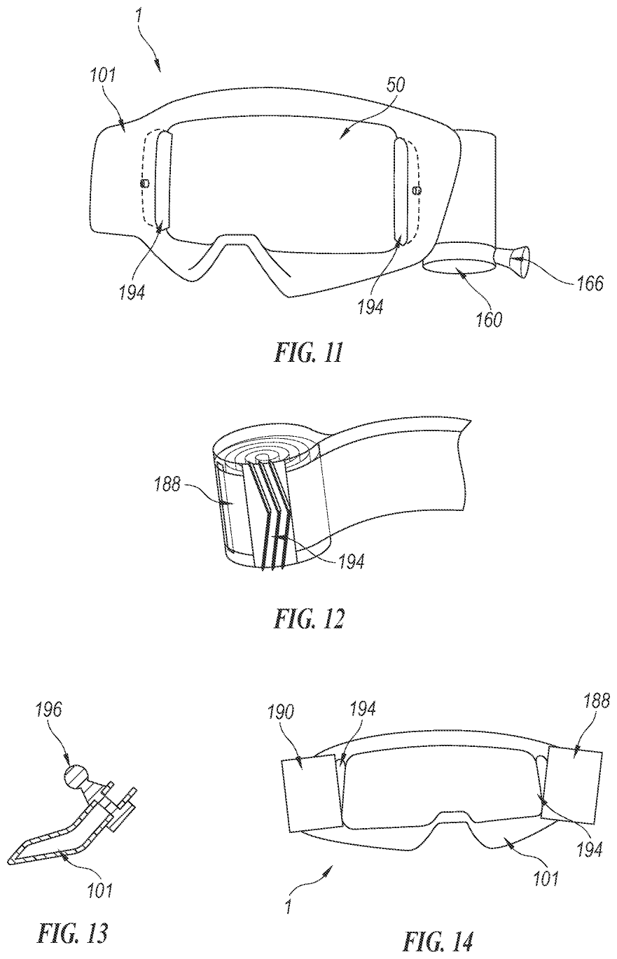

As noted above, the example lens cleaning system 100 is shown as a roll-off system. As shown in FIGS. 1-14, the roll-off system 100 includes a first canister, shown as intake canister 120, a second canister, shown as supply canister 140, an actuator mechanism, shown as actuation system 160, and a film, shown as film 180 (see FIG. 10). The intake canister 120 and the supply canister 140 may at least partially be integrally formed (e.g., molded, permanently fixed, non-detachable, etc.) with the frame 20. For example, the intake canister 120 and the supply canister 140 may form a part of and/or be embedded within the frame 20. Additionally and/or alternatively, the intake canister 120 and the supply canister 140 are separately coupled to the frame 20 and/or to the lens 50. For example, the intake canister 120 and/or the supply canister 140 may be adhesively secured or otherwise mechanically fastened or mounted to the frame 20 and/or the lens 50. Thus, the intake canister 120 and/or the supply canister 140 may extend outwardly from a first side (or front surface) of the frame 20 and/or the lens 50, making it more likely that debris contacts and/or becomes packed onto the intake canister 120 and/or the supply canister 140 of the lens cleaning system 100. In some implementations, the intake canister 120 and/or the supply canister 140 may additionally and/or alternatively extend from a second side (or rear surface facing the wearer) of the frame 20 and/or the lens 50, laterally outwardly from lateral sides of the frame 20 (or outriggers as noted below), or upwardly, downwardly, and/or inwardly from the frame 20, and/or the like. In such implementations, even if the intake canister 120 and/or the supply canister 140 extends from a second side (or rear surface facing the wearer) of the frame 20 and/or the lens 50, laterally outwardly from lateral sides of the frame 20 (or outriggers as noted below), or upwardly, downwardly, and/or inwardly from the frame 20, there is a risk that debris contacts and/or becomes packed onto the intake canister 120 and/or the supply canister 140 of the lens cleaning system 100. The protective cover 101 described herein helps to prevent or limit debris from contacting and/or becoming packed onto the intake canister 120 and/or the supply canister 140, among other components of the lens cleaning system 100.

Additionally and/or alternatively, the intake canister 120 and the supply canister 140 are separately coupled to outriggers, which form a part of and/or are coupled to the frame 20 for coupling of the headband to the eyewear. The film 180 may be a continuous film that extends across a surface of the lens 50 between the intake canister 120 and the supply canister 140.

The intake canister 120 may include a first housing 112, a first spindle, and a first cover 113. The first housing 112 may define a first cavity (not shown) configured to receive the first spindle. The first cover 113 is configured to enclose the first cavity of the intake canister 120. The supply canister 140 may include a second housing 122, a second spindle, and a second cover 123. The second housing 122 may define a second cavity (not shown) configured to receive the second spindle. The second cover 123 is configured to enclose the second cavity of the supply canister 140.

In some implementations, the film 180 extends between the intake canister 120 and the supply canister 140 (see FIG. 10). For example, a first portion of the film 180 may extend within the second cavity of the supply canister 140 and around the second spindle, and a second portion of the film 180 may extend within the first cavity of the intake canister 120 and around the first spindle. In some implementations, a rear portion of the film 180 is configured to be disposed along a first side or surface of the lens 50 and/or a front portion of the film 180 is configured to be disposed along a second side or surface of the lens 50 opposite the first side.

As noted above, the first and second cavities may be accessed such that the film 180 is deposited into at least one of the intake canister 120 and the supply canister 150. Thus, the film 180 may be stored and/or otherwise secured within at least one of the intake canister 120 and/or the supply canister 140.

The film 180 may be transferred from the supply canister 140 to the intake canister 120. For example, the second cavity of the supply canister 140 may store a roll of the film 180 and the first cavity of the intake canister 120 may receive a strip of the film 180 from the roll of the film 180 stored within the supply canister 140. The actuation system 160 may facilitate selectively dispensing the strip of the film 180 from the roll of the film 180 from within the second cavity of the supply canister 140, across a surface, shown as front surface 52, of the lens 50, to the first cavity of the intake canister 120.

As shown in FIGS. 1-3 and 8-11, the actuation system 160 is positioned to couple to the intake canister 120. The actuation system 160 includes a housing, shown as actuator housing 162, a pull-cord, and a knob, shown as pull-knob 166. The actuator housing 162 may include a rotational member (e.g., a torsional spring, etc.) that facilitates unwinding (e.g., in response to a wearer pulling on the pull-knob, automatically, etc.) and winding the pull-string (e.g., in response to the wearer releasing the pull-knob 166 after being pulled, automatically, etc.). The actuation system 160 may dispense a new strip portion of the film 180 (e.g., a clean portion, an unused portion, etc.) from the roll of the film 180 stored in second cavity of the supply canister 140. The new strip portion of the film 180 may be dispensed, and extend across the front surface 52 of the lens 50. The new strip portion of the film may also extend into the first cavity of the intake canister 120, for example, in response to a wearer of the eyewear 10 pulling on the pull-knob 166. The intake canister 120 is thereby configured to receive and store the old strip portion of the film 180 (e.g., the used portion, the dirty portion, roll up the film strip onto a spindle, etc.). In some implementations, the frame 20 and/or lens 50 includes or defines an extension or scraper element positioned to engage the strip of the film 180 as the film 180 is fed into the intake canister 120 and/or the supply canister 140. When the strip of film is fed into the intake canister 120, for example, the extension or scraper element may remove particles (e.g., debris, mud, dirt, particles, etc.) from the film 180 as the film 180 enters the intake canister 120. In some implementations, the roll-off system 100 is a continuous roll-off system including a single, continuous loop of film similar to that disclosed in U.S. patent application Ser. No. 15/182,018 (Publication No. US 2017/0354540), filed on Jun. 14, 2016, which is incorporated herein by reference in its entirety.

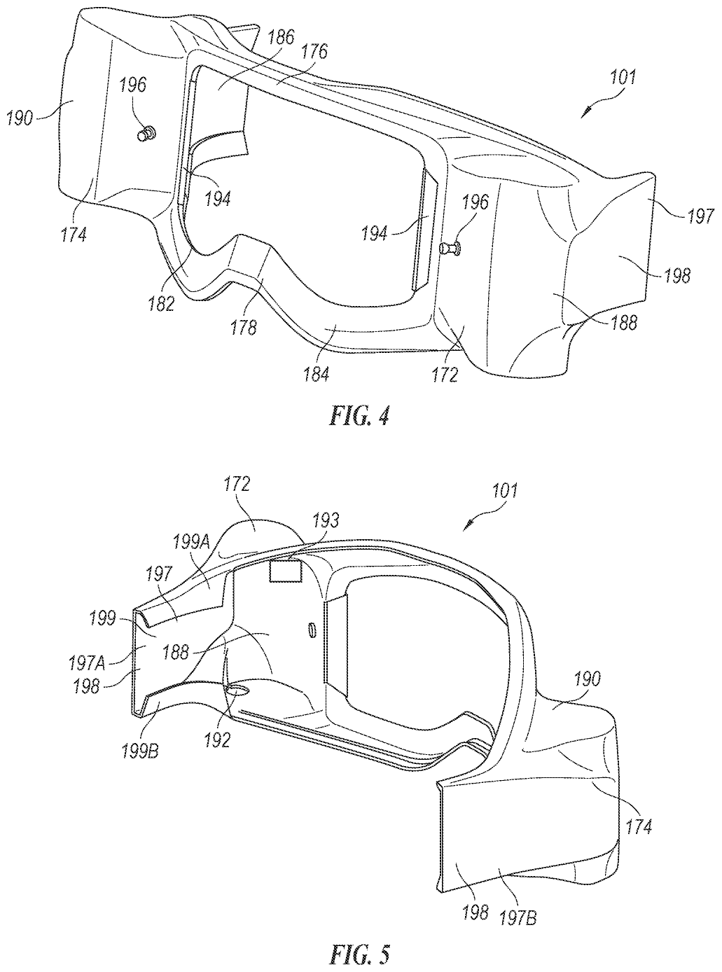

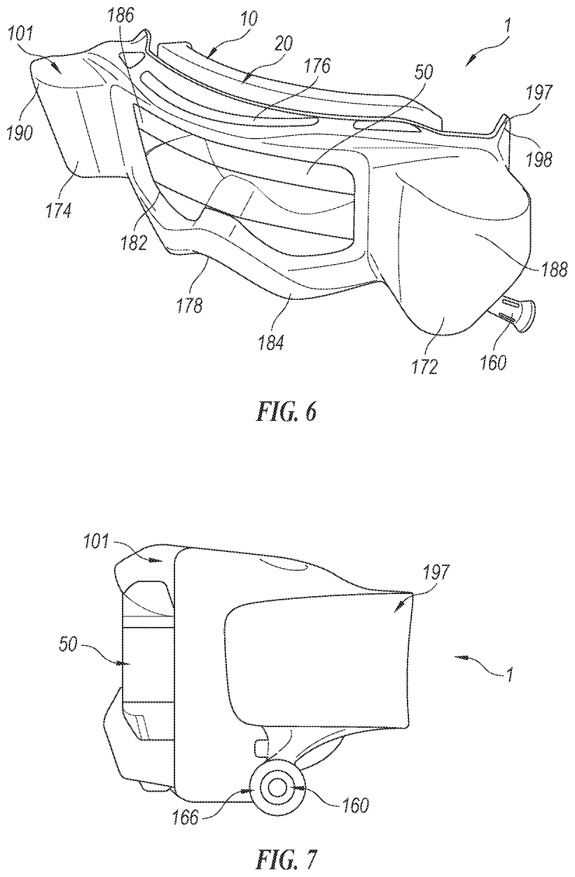

FIGS. 1 and 2 illustrate an example of the protective cover 101 coupled to the eyewear 10. FIG. 3 illustrates an exploded view of the eyewear system 1, consistent with implementations of the current subject matter. FIGS. 4 and 5 illustrate an example of the protective cover 101, consistent with implementations of the current subject matter. FIGS. 6-10 and 11-14 illustrate examples of the eyewear system 1 including the protective cover 101 and the eyewear 10, consistent with implementations of the current subject matter, which includes the same features as the eyewear system 1 illustrated in FIGS. 1-5.

The protective cover 101 may help to reduce, or prevent failure of the lens cleaning system 100 of the eyewear 10, during use. For example, the shape, size, material, and/or the like of the protective cover 101 allows the protective cover to couple to, enclose, and/or tightly seal around at least a portion of the lens cleaning system 100 and at least the first side (e.g., outwardly facing side) of the frame 20. Thus, the protective cover 101 may reduce the surface area of the frame and/or the lens cleaning system that is exposed to mud, water, or other debris. This makes it safer for the wearer of the eyewear 10, as the protective cover 101 helps to reduce the likelihood of failure of the lens cleaning system 100 to properly clean the lens 50.

As noted above, the eyewear 10 may include the frame 20, the lens 50 disposed within the frame 20, and the lens cleaning system 100. Also, as described herein, the lens cleaning system 100 may include a roll-off system including a first canister or first portion, such as the intake canister 120, a second canister or second portion, such as the supply canister 140, and/or an actuation system 160 for cleaning the lens 50. The protective cover 101 may couple to the frame 20 to cover at least a first side of the frame 20 and at least a portion of the lens cleaning system 100, such as the first portion 120 and/or the second portion 140 of the lens cleaning system 100. Thus, the protective cover 101 may prevent or limit debris from contacting and/or otherwise packing on the lens cleaning system 100, making it easier for the lens cleaning system 100 to clean the lens 50 and reducing the likelihood of failure or jamming of the lens cleaning system 100.

In some implementations, the protective cover 101 forms a unitary cover that is configured to be coupled to the eyewear 10 (see FIGS. 1-14). For example, the protective cover 101 may include a first cover end portion 172, a second cover end portion 174, a top cover portion 176, and/or a bottom cover portion 178. The top cover portion 176 may extend from the first cover end portion 172 to the second cover end portion 174. The bottom cover portion 178 may also extend from the first cover end portion 172 to the second cover end portion 174. In some implementations, the first cover end portion 172, the second cover end portion 174, the top cover portion 176, and/or the bottom cover portion 178 together surround an opening, which defines a lens opening 182. The lens opening 182 allows unobstructed and/or minimally obstructed access to the lens 50. The first cover end portion 172, the second cover end portion 174, the top cover portion 176, and the bottom cover portion 178 may correspond to and/or cover respectively portions of the frame 20.

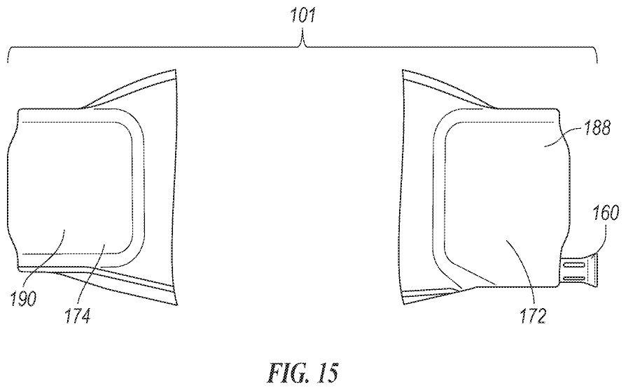

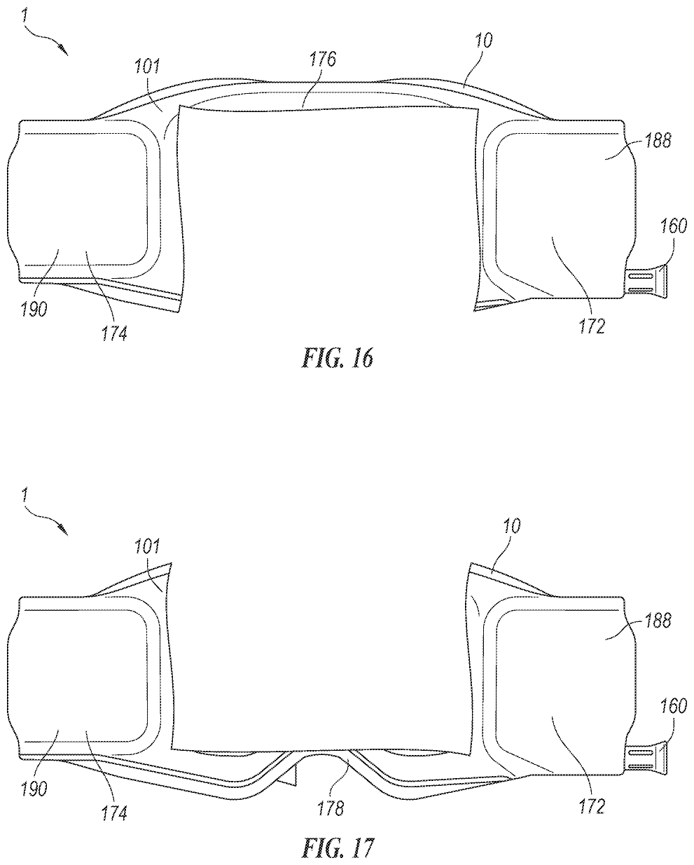

Additionally and/or alternatively, the protective cover 101 is formed of multiple cover portions. For example, as shown in FIG. 15, the first cover end portion 172 and the second cover end portion 174 may each for individual and separate portions of the protective cover 101. In this example, the separate first cover end portion 172 and the separate second cover end portion 174 may each be configured to cover and/or couple to a respective portion of the eyewear 10, such as respective portions of the frame 20 and/or the lens cleaning system 100. For example, the separate first cover end portion 172 may cover and/or couple to at least a first portion of the lens cleaning system 100 such as the intake canister 120, and the separate second cover end portion 174 may cover and/or couple to at least a second portion of the lens cleaning system 100 such as the supply canister 140. Additionally and/or alternatively, the first cover end portion 172 and the second cover end portion 174 connected be either the top cover portion 176 (see FIG. 16) or the bottom cover portion 178 (see FIG. 17). Such configurations help to reduce materials and/or may make the eyewear system 1 more lightweight, thereby improving the comfort of the wearer.

In some implementations, the protective cover 101 includes an outer side 184 and an inner side 186. The outer side 184 is configured to face away from the eyewear 10 when the protective cover 101 is coupled to the eyewear 10. The inner side 186 is configured to face towards, contact and/or couple to the eyewear 10. In some implementations, the protective cover 101 has a shape that corresponds to a shape of at least a portion of the eyewear 10. For example, in some implementations, the inner side 186 has a shape that corresponds to a shape of at least the first side (e.g., outwardly facing side and/or lateral sides) of the frame 20 and/or a shape of the lens cleaning system 100.

As shown in FIGS. 4 and 5, for example, the inner side 186 includes a first cleaning system receiver 188 and a second cleaning system receiver 190. The first cleaning system receiver 188 and a second cleaning system receiver 190 may be positioned on opposite sides of the protective cover 101. For example, the first cleaning system receiver 188 may be positioned at the first cover end portion 172 and the second cleaning system receiver 190 may be positioned at the second cover end portion 174, opposite the first cleaning system receiver 188.

The first cleaning system receiver 188 may include a shape that corresponds to a first portion, such as the intake canister 120, of the lens cleaning system 100. This allows the first cleaning system receiver 188 to cover and/or tightly seal around the first portion of the lens cleaning system 100. The second cleaning system receiver 190 may include a shape that corresponds to a second portion, such as the supply canister 140, of the lens cleaning system 100. This allows the second cleaning system receiver 190 to cover and/or tightly seal around the second portion of the lens cleaning system 100.

The first cleaning system receiver 188 and/or the second cleaning system receiver 190 may each define a recessed portion of the protective cover 101. Each recessed portion of the protective cover 101 may be shaped to receive and/or correspond to at least the first and second portions of the lens cleaning system 100, such as the intake and supply canisters 140, 120.

In some implementations, while the first cleaning system receiver 188 and/or the second cleaning system receiver 190 each define a recessed portion of the protective cover 101 along the inner side 186 of the protective cover 101, the first cleaning system receiver 188 and/or the second cleaning system receiver 190 may each define a protrusion that extends outwardly from an outer surface of the protective cover 101 along the outer side 184.

In some implementations, the protective cover 101 includes a slot and/or opening 192. The slot and/or opening 192 may allow for at least a portion of the actuation system 160 to be accessed, to actuate the lens cleaning system 100 to clean the lens 50. For example, the slot and/or opening 192 may allow access to the pull-knob 166. As shown in FIG. 5, the slot and/or opening 192 may allow the pull-knob 166 to extend through the protective cover 101 to be easily accessed by the wearer of the eyewear 10. In some implementations, the protective cover 101 covers at least a portion of the actuation system 160, such as the pull-knob 166 to further reduce entry points for debris to contact and/or otherwise disrupt the lens cleaning system 100 (see FIG. 7).

In some implementations, the protective cover 101 includes a scraper 194 and/or a tear-off post 196 (see FIGS. 4-5 and 10-14). The scraper 194 may include one or more blades, such as raised blades, to help clean the film 180. For example, the one or more blades of the scraper 194 may be made of silicone or another material that helps to remove debris from the film 180. In some implementations, the scraper 194 removes debris from the film 180 without tearing the film 180. The scraper 194 may define a rip-off interface configured to facilitate detachably coupling a rip-off film sheet of the film 180.

The scraper 194 may be separately coupled to and/or formed with the protective cover 101. For example, the scraper 194 may be built into the protective cover 101. The scraper 194 may include one, two, three, four, or more scrapers 194. For example, the scraper 194 may include a first scraper positioned at one lateral end of the lens opening 182 adjacent the first cover end portion 172 and/or a second scraper positioned at an opposite lateral end of the lens opening 182 adjacent the second cover end portion 174.

The scraper 194 is positioned to engage at least a portion of the film 180 to remove particles (e.g., debris, mud, dirt, larger particles, etc.) from the film 180. In some implementations, the scraper 194 includes a cleaning element to remove debris particles, such as finer debris particles, from the film 180 and/or the lens 50, and/or to dry the film 180 and/or the lens 50, such as when the film 180 cycles from the supply canister 140 to the intake canister 120. In some implementations, the cleaning element includes a drying element (e.g., a squeegee, an absorbent pad, a microfiber cloth, a brush, felt, and the like) configured to dry liquid (e.g., water, snow, rain, etc.) and/or smooth the liquid that may be disposed on the film 180. In some implementations, the cleaning element includes a scraper element or brush element configured to remove finer particles. In some implementations, the scraper 194 dispenses a liquid, such as a liquid cleaning solution, to help clean the film 180 and/or the lens 50. The scraper 194 helps to remove debris from the film 180 before and/or after the film 180 enters and/or exists one or both of the supply canister 140 and the intake canister 120. In some implementations, such as the example shown in FIG. 14, the scrapers 194 may be angled to better remove debris from the film 180 before and/or after the film 180 enters and/or exists one or both of the supply canister 140 and the intake canister 120.

In some implementations, the protective cover 101 includes the tear-off post 196. In some implementations, the tear-off post 196 extends from the outer side 184 of the protective cover 101. Additionally and/or alternatively, the tear-off post 196 is molded and/or over-molded into the protective cover 101. Additionally and/or alternatively, the tear-off post 196 is separately coupled to the protective cover 101. For example, the tear-off post 196 may be pressed, clipped, snapped, and/or the like onto the protective cover 101. Additionally and/or alternatively, the tear-off post 196 may be coupled to and/or extend from the frame 20 of the eyewear 10. In such implementations, the protective cover 101 may include an opening that allows the tear-off post 196 of the eyewear 10 to pass through and be accessed by the wearer.

The tear-off post 196 may include one, two, three, four, or more tear-off post 196. For example, the tear-off post 196 may include a first tear-off post positioned at one lateral end of the lens opening 182 adjacent the first cover end portion 172 and/or a second tear-off post positioned at an opposite lateral end of the lens opening 182 adjacent the second cover end portion 174.

The tear-off post 196 may hold a small stack of tear-off film sheets to protect the roll-off system or lens cleaning system 100, such as during a first lap of a race, when the riders are more often located close together. The tear-off post 196 may form a part of a rip-off system that is coupled to and/or forms a part of the protective cover 101 and/or the frame 20. Thus, in some implementations, the eyewear system 1 may include a hybrid lens cleaning system 100 that includes both a roll-off system (positioned on or coupled to the frame 20) and a rip-off system (positioned on or coupled to the protective cover 101, the frame 20, and/or the like). The rip-off system, including the tear off posts 196 may facilitate detachably coupling one or more rip-off film sheets across the lens 50 (e.g., from a front of the lens 50) and the quick removal thereof.

As described herein, the protective cover 101 may be coupled to the eyewear 10, such as to the frame 20. For example, the eyewear 10 may be provided and the protective cover 101 may be coupled to the eyewear 10. The protective cover 101 may be coupled to the eyewear 10 via an adhesive, a snap-fit arrangement, a mechanical fastener, a stretch and/or compression fit, a shrink fit, and/or another attachment mechanism. To securely attach the protective cover 101 to the frame 20, the protective cover 101 includes one or more attachment features 198. The one or more attachment features 198 may include one or more tabs 197. The one or more tabs 197 may include on, two, three, four, or more tabs. The tabs 197 may extend from at least a portion of the protective cover 101. For example, a first tab 197A may extend from the first cover end portion 172 and a second tab 1978 may extend from the second cover end portion 174. The tabs 197 may extend away from the first side (e.g., outwardly facing side) of the protective cover 101 and/or may extend from respective recessed portions of the protective cover 101. The tabs 197 may be configured to cover lateral sides of the eyewear 10 when the protective cover 101 is coupled to the eyewear 10.

The tabs 197 may help a wearer to more easily couple and/or de-couple the protective cover from the eyewear 10. For example, the tabs 197 provide a greater surface area for a wearer to grasp the protective cover 101. The tabs 197 may include a track 199. The track 199 may help to more securely and/or easily attach the protective cover 101 to the eyewear 10. For example, the track 199 may include an upper track portion 199A and a lower track portion 1998. The upper track portion 199A and the lower track portion 1998 of the track 199 of each tab 197 may be spaced inwardly from the inner side 186 of the protective cover 101. The track 199 is configured to slide over at least a portion of the eyewear 10, such as opposing lateral sides of the eyewear 10. The upper and lower track portions 199A, 1998 of each track 199 is configured to slide over and/or snap over at least the lateral sides of the eyewear 10 to help secure the protective cover 101 to the eyewear 10. The tracks 199 may help to prevent movement (e.g., forward-rearward movement and/or lateral movement) of the protective cover 101 relative to the eyewear 10. This helps to properly position the protective cover 101 relative to the eyewear 10 to further prevent or reduce the likelihood that debris will contact and/or become lodged onto the lens cleaning system 100 of the eyewear 10. Thus, the protective cover 101 helps to protect the lens cleaning system 100 of the eyewear 10 during use.

In some implementations, the one or more attachment features 198 includes an adhesive lining and/or another adhesive 193 that secures the inner side 186 of the protective cover 101 to the frame 20, such as the first side of the frame 20, the lateral sides of the frame 20, the second side of the frame 20, the lens cleaning system 100, such as the intake canister 120 and the supply canister 140, and/or another portion of the eyewear 10. The adhesive helps to properly position the protective cover 101 relative to the eyewear 10 and/or seal the protective cover 101 around the frame 20 and/or the lens cleaning system 100 to further prevent or reduce the likelihood that debris will contact and/or become lodged onto the lens cleaning system 100 of the eyewear 10.

In some implementations, the protective cover 101 includes a flexible material. In some implementations, the protective cover 101 includes a silicone, rubber, or other material. For example, the protective cover 101 includes a rubber, silicone, and/or other flexible material that stretches over at least the first side of the frame and/or the lens cleaning system for a tight fit and/or seal. The material of the protective cover 101 may help to secure (e.g., sealably secure) the protective cover 101 to the eyewear 10 to prevent or reduce the likelihood that debris will contact and/or become lodged onto the lens cleaning system 100 of the eyewear 10. In some implementations, the protective cover 101 include rigid and/or semi-rigid materials. Additionally and/or alternatively, the protective cover 101 may including a coating, such as a non-stick coating, that helps to prevent debris from sticking to the protective cover 101 and/or the eyewear 10. Thus, the protective cover 101 described herein helps to improve the performance of the eyewear 10 and in particular, the lens cleaning system 100, extends the usable life of the lens cleaning system 100, and/or protects the wearer, making it safer to use the eyewear 10 during activities, such as races or competitions where it is difficult or unsafe for the wearer to stop and/or manually clean the eyewear 10.

In some implementations, the eyewear 10 (e.g., the frame 20) additionally and/or alternatively includes one or more wipers. The one or more wipers may be positioned at various locations along the frame 20, such as along first and/or second lateral end portions of the frame 20. In some implementations, the one or more wipers may be positioned at various locations along the lens 50, such as along first and/or second lateral sides of the lens 50. In some implementations, the one or more wipers are stationary, such as relative to the frame 20 and/or the lens 50. In such implementations, the one or more wipers may clean debris or other substances from the film 180 and/or lens 50, such as when the film is rolled, pulled, and/or otherwise moved across the lens 50. In other implementations, the one or more wipers may pivot, rotate, or may otherwise move across at least a portion of the front surface 52 of the lens 50 to clean debris or other substances from the front surface 52 of the lens 50 and/or from the film 180. In some implementations, the one or more wipers (e.g., automatically or via manual actuation) move across the front surface 52 before the film 180 is applied to the lens 50. The one or more wipers may further improve the cleaning of the eyewear (e.g., via increased speed, performance, and efficiency).

In some implementations, the eyewear 10 additionally and/or alternatively includes an attachment system for a light (e.g., a flashlight, an LED light, etc.). The attachment system for the light may be positioned on the first or second end portions of the frame 20, the first and/or second canisters of the roll-off system 100, and/or other positions. The attachment system for the light may be configured to receive and hold a light-emitting device (e.g., a flashlight, LEDs, etc.) on the eyewear 10 to illuminate a visible area in front of and/or to the sides of the wearer.

In some implementations, the eyewear 10 additionally and/or alternatively includes an attachment system for a camera (e.g., an action camera, a still-photography camera, a GoPro®, etc.). The attachment system for the camera may be positioned on the right or left portions of the frame 20 and/or the first and/or second canisters of the roll-off system 100. In some implementations, the attachment system for the camera is positioned on one side of the eyewear 10 to receive and hold a camera device for capturing photos and/or video of the wearer performing some action (e.g., riding a dirt bike, etc.). In other implementations, the attachment system for the camera is positioned on both sides of the eyewear 10 to receive and hold two camera devices to the eyewear 10. According to an exemplary implementation, the eyewear 10 having two camera devices attached thereto facilitates three-dimensional and/or virtual filming of the wearer's actions.

In some implementations, the eyewear 10 additionally or alternatively includes an attachment system for an integrated heads up display (HUD). The attachment system for the HUD may be positioned on the eyewear 10. The attachment system for the HUD may be configured to receive and hold a HUD on the eyewear 10 to provide various information across the lens 50 for viewing by the wearer of the eyewear 10 (e.g., directions, notifications, vehicle information, etc.).

Examples of embodiments of the current technology include, but are not limited to, the following:

1. A protective cover for an eyewear having a lens cleaning system and a frame, the protective cover comprising:
a first end portion;
a second end portion;
a lens opening configured to allow access to a lens of the eyewear, the lens opening positioned laterally between the first end portion and the second end portion;
an outer side;
an inner side, the inner side comprising a shape that corresponds to a shape of at least a first side of the frame of the eyewear and at least a portion of the lens cleaning system, the inner side further comprising:
a first cleaning system receiver configured to cover a first portion of the lens cleaning system of the eyewear, the first cleaning system receiver positioned at the first end portion; and
a second cleaning system receiver configured to cover a second portion of the lens cleaning system of the eyewear, the second cleaning system receiver positioned at the second end portion;
wherein the protective cover is configured to couple to the frame;
wherein the protective cover is configured to enclose at least a first side of the frame and at least the portion of the lens cleaning system, the first side extending across a surface of the frame configured to face away from the wearer when worn; and
wherein the protective cover is configured to reduce a failure of the lens cleaning system due to debris contacting the lens cleaning system.

2. The protective cover of claim 1, wherein the lens cleaning system comprises a roll-off system.

3. The protective cover of claim 2, wherein the first portion of the lens cleaning system is a first portion of a first canister, the first canister configured to store at least a first portion of a film for cleaning the lens of the eyewear; and wherein the second portion of the lens cleaning system is a second portion of a second canister, the second canister configured to store at least a second portion of the film.

4. The protective cover of claim 1, further comprising a scraper configured to tear a film of the lens cleaning system.

5. The protective cover of claim 4, wherein the scraper comprises a first scraper positioned on the first end portion; and a second scraper positioned on the second end portion, the first scraper and the second scraper each defining a rip-off interface configured to facilitate detachably coupling a rip-off film sheet across the lens received of the eyewear.

6. The protective cover of claim 4, wherein the scraper comprises a plurality of raised blades.

7. The protective cover of claim 1, further comprising:
a top portion; and
a bottom portion, opposite the top portion;
wherein the top portion extends from the first end portion to the second end portion;
wherein the bottom portion extends from the first end portion to the second end portion; and
wherein the top portion, the bottom portion, the first end portion, and the second end portion are configured to surround the lens opening of the cover.

8. The protective cover of claim 1, further comprising a flexible material, the flexible material comprising silicone.

9. The protective cover of claim 1, wherein the protective cover is configured to couple to the frame of the eyewear via one or more of an adhesive, a snap-fit arrangement, and a mechanical fastener.

10. The protective cover of claim 1, wherein the eyewear comprises a goggle.

11. The protective cover of claim 1, further comprising one or more attachment features for securing the protective cover to the eyewear, the one or more attachment features comprising a tab comprising a track configured to position the protective cover relative to the eyewear.

12. An eyewear system, comprising:
an eyewear comprising:
a frame;
a lens disposed within the frame;
a lens cleaning system for cleaning the lens;
a first side extending across the frame;
a second side opposite the first side, the second side configured to contact a face of a wearer of the eyewear when worn; and
a protective cover comprising:
a first end portion;
a second end portion;
a lens opening configured to allow access to the lens, the lens opening positioned laterally between the first end portion and the second end portion;
an outer side;
an inner side, the inner side comprising a shape that corresponds to a shape of at least the first side of the frame and at least the portion of the lens cleaning system, the inner side further comprising:
   a first cleaning system receiver configured to cover a first portion of the lens cleaning system, the first cleaning system receiver positioned at the first end portion; and
   a second cleaning system receiver configured to cover a second portion of the lens cleaning system, the second cleaning system receiver positioned at the second end portion;
wherein the protective cover is coupled to the frame;
wherein the protective cover is configured to enclose at least the first side and at least the portion of the lens cleaning system; and
wherein the protective cover is configured to reduce a failure of the lens cleaning system due to debris contacting the lens cleaning system.

13. The eyewear system of claim 12, wherein the lens cleaning system comprises a roll-off system.

14. The eyewear system of claim 13, wherein the first portion of the lens cleaning system is a first portion of a first canister, the first canister configured to store at least a first portion of a film for cleaning the lens of the eyewear; and
wherein the second portion of the lens cleaning system is a second portion of a second canister, the second canister configured to store at least a second portion of the film.

15. The eyewear system of claim 12, wherein the protective cover further comprises a scraper configured to tear a film of the lens cleaning system.

16. The eyewear system of claim 15, wherein the scraper comprises a first scraper positioned on the first end portion; and a second scraper positioned on the second end portion, the first scraper and the second scraper each defining a rip-off interface configured to facilitate detachably coupling a rip-off film sheet across the lens.

17. The eyewear system of claim 15, wherein the scraper comprises a plurality of raised blades.

18. The eyewear system of claim 12, wherein the protective cover further comprises:
a top portion; and
a bottom portion, opposite the top portion;
wherein the top portion extends from the first end portion to the second end portion;
wherein the bottom portion extends from the first end portion to the second end portion; and
wherein the top portion, the bottom portion, the first end portion, and the second end portion are configured to surround the lens opening of the cover.

19. The eyewear system of claim 12, wherein the protective cover comprises a flexible material.

20. The eyewear system of claim 12, wherein the protective cover comprises a silicone material.

21. The eyewear system of claim 12, wherein the protective cover is coupled to the frame of the eyewear via one or more of an adhesive, a snap-fit arrangement, and a mechanical fastener.

22. The eyewear system of claim 12, wherein the eyewear comprises a goggle.

23. An method of reducing a failure of the lens cleaning system of an eyewear due to debris contacting the lens cleaning system, the method comprising:
providing the eyewear, the eyewear comprising:
a frame;
a lens;
a lens cleaning system for cleaning the lens;
a first side extending across the frame;
a second side opposite the first side, the second side configured to contact a face of a wearer of the eyewear when worn; and
coupling a protective cover to the eyewear such that the protective cover encloses at least the first side of the eyewear and at least a portion of the lens cleaning system, the protective cover comprising:
   a first end portion;
   a second end portion;
   a lens opening configured to allow access to the lens, the lens opening positioned laterally between the first end portion and the second end portion;
   an outer side;
   an inner side, the inner side comprising a shape that corresponds to a shape of at least the first side of the frame and at least the portion of the lens cleaning system, the inner side further comprising:
   a first cleaning system receiver configured to cover a first portion of the lens cleaning system, the first cleaning system receiver positioned at the first end portion; and
   a second cleaning system receiver configured to cover a second portion of the lens cleaning system, the second cleaning system receiver positioned at the second end portion.

24. An eyewear system, comprising
a goggle having a frame and a lens carried by the frame;
a lens cleaning system coupled to the frame and positioned adjacent to the lens; and
a protective cover releasably coupled to the goggle and the lens cleaning system, the protective cover comprising:
opposing first and second end portions;
a lens opening laterally between the first end portion and the second end portion, the lens opening configured to allow access to the lens of the eyewear for visibility through the lens, the lens cleaning system and the lens opening;
an outer side facing away from the frame;
an inner side facing toward the frame, the inner side having a contour that corresponds to a shape of at least a first side of the frame and at least a portion of the lens cleaning system, the inner side further comprising:
a first cleaning system receiver configured to cover a first portion of the lens cleaning system, the first cleaning system receiver positioned at the first end portion; and
a second cleaning system receiver configured to cover a second portion of the lens cleaning system of the eyewear, the second cleaning system receiver positioned at the second end portion;
wherein the protective cover is configured to releasably couple to the frame and movable between an installed position on the frame and a separated position removed from the frame;
wherein the protective cover in the installed position encloses at least a first side of the frame and at least the portion of the lens cleaning system, the first side extending across a surface of the frame configured to face away from the wearer when worn; and
wherein the protective cover in the installed position is configured to block debris from engaging the lens cleaning system during use of the eyewear system.

25. The eyewear system of claim 24, wherein the lens cleaning system comprises a roll-off system.

26. The eyewear system of claim 24, wherein the first portion of the lens cleaning system comprises a first canister configured to store at least a first portion of a film for extending across the lens of the eyewear; and the second portion of the lens cleaning system comprises a second canister configured to store at least a second portion of the film.

27. The eyewear system of claim 24, wherein the protective cover has comprises a scraper configured to tear a film of the lens cleaning system.

28. The eyewear system of claim 24, wherein the scraper comprises a first scraper positioned on the first end portion; and a second scraper positioned on the second end portion, the first scraper and the second scraper each defining a rip-off interface configured to facilitate detachably coupling a rip-off film sheet across the lens received of the eyewear.

29. The eyewear system of claim 27, wherein the scraper comprises a plurality of raised blades.

30. The eyewear system of claim 24, wherein the frame of the goggle comprises opposing top and bottom portions, wherein the top portion extends from the first end portion to the second end portion, and the bottom portion extends from the first end portion to the second end portion, and wherein the top portion, the bottom portion, the first end portion, and the second end portion are configured to surround the lens opening of the protective cover.

31. The eyewear system of claim 24, wherein the protective cover is releasably coupled to the goggle by an adhesive, a snap-fit arrangement, or a mechanical fastener.

32. The protective cover of claim 24, further comprising one or more attachment features for securing the protective cover to the goggle, the one or more attachment features comprising a tab comprising a track configured to position the protective cover relative to the eyewear.

33. A protective eyewear system, comprising
an eyewear having a frame, a lens coupled to the frame, and a lens cleaning system coupled to the frame and positioned adjacent to the lens; and
a protective cover coupled to the eyewear, the protective cover comprising: opposing first and second end portions;
   a lens opening laterally between the first end portion and the second end portion, the lens opening configured to allow access to the lens of the eyewear for visibility through the lens, the lens cleaning system and the lens opening;
   an outer side facing away from the frame;
   an inner side facing toward the frame, the inner side having a shape that corresponds to a shape of at least a first side of the frame and at least a portion of the lens cleaning system, the inner side further comprising:
   a first cleaning system receiver configured to cover a first portion of the lens cleaning system, the first cleaning system receiver positioned at the first end portion; and
   a second cleaning system receiver configured to cover a second portion of the lens cleaning system of the eyewear, the second cleaning system receiver positioned at the second end portion;
   wherein the protective cover is configured to releasably couple to the frame and movable between an installed position on the frame and a separated position removed from the frame;
   wherein the protective cover in the installed position encloses at least a first side of the frame and at least the portion of the lens cleaning system, the first side extending across a surface of the frame configured to face away from the wearer when worn; and
wherein the protective cover in the installed position is configured to block debris from engaging the lens cleaning system during use of the eyewear system.

34. The protective eyewear system of claim 33, wherein the lens cleaning system comprises a roll-off system.

35. The protective eyewear system of claim 33, wherein the first portion of the lens cleaning system comprises a first canister configured to store at least a first portion of a film for extending across the lens of the eyewear; and the second portion of the lens cleaning system comprises a second canister configured to store at least a second portion of the film.

36. The protective eyewear system of claim 33, wherein the protective cover has a first scraper positioned on the first end portion; and a second scraper positioned on the second end portion, the first scraper and the second scraper each defining a rip-off interface configured to facilitate detachably coupling a rip-off film sheet across the lens received of the eyewear.

37. The protective eyewear system of claim 33, wherein the frame of the eyewear comprises opposing top and bottom portions, wherein the top portion extends from the first end portion to the second end portion, and the bottom portion extends from the first end portion to the second end portion, and wherein the top portion, the bottom portion, the first end portion, and the second end portion are configured to surround the lens opening of the protective cover.

38. The protective eyewear system of claim 33, wherein the protective cover comprises one or more attachment features for securing the protective cover to the eyewear, the one or more attachment features comprising a tab comprising a track configured to position the protective cover relative to the frame.

39. A protective cover for an eyewear having a lens cleaning system and a frame, the protective cover comprising:
a first end portion;
a second end portion;
a lens opening configured to allow access to a lens of the eyewear, the lens opening positioned laterally between the first end portion and the second end portion;
an outer side;
an inner side, the inner side comprising a shape that corresponds to a shape of at least a first side of the frame of the eyewear and at least a portion of the lens cleaning system, the inner side further comprising:
a first cleaning system receiver configured to cover a first portion of the lens cleaning system of the eyewear, the first cleaning system receiver positioned at the first end portion; and
a second cleaning system receiver configured to cover a second portion of the lens cleaning system of the eyewear, the second cleaning system receiver positioned at the second end portion;
wherein the protective cover is configured to couple to the frame;
wherein the protective cover is configured to enclose at least a first side of the frame and at least the portion of the lens cleaning system, the first side extending across a surface of the frame configured to face away from the wearer when worn; and
wherein the protective cover is configured to reduce a failure of the lens cleaning system due to debris contacting the lens cleaning system.

40. The protective cover of claim 39, wherein the first portion of the lens cleaning system is a first portion of a first canister, the first canister configured to store at least a first portion of a film for cleaning the lens of the eyewear; and wherein the second portion of the lens cleaning system is a second portion of a second canister, the second canister configured to store at least a second portion of the film.

41. The protective cover of claim 39, further comprising a scraper configured to tear a film of the lens cleaning system.

42. The protective cover of claim 41, wherein the scraper comprises a plurality of raised blades.

43. The protective cover of claim 39, further comprising:
a top portion; and
a bottom portion, opposite the top portion;
wherein the top portion extends from the first end portion to the second end portion;
wherein the bottom portion extends from the first end portion to the second end portion; and
wherein the top portion, the bottom portion, the first end portion, and the second end portion are configured to surround the lens opening of the cover.

44. The protective cover of claim 39, further comprising one or more attachment features for securing the protective cover to the eyewear, the one or more attachment features comprising a tab comprising a track configured to position the protective cover relative to the eyewear.

45. An eyewear system, comprising:
an eyewear comprising:
   a frame;
   a lens disposed within the frame;
   a lens cleaning system for cleaning the lens;
   a first side extending across the frame;
   a second side opposite the first side, the second side configured to contact a face of a wearer of the eyewear when worn; and
a protective cover removably attachable to the eyewear, the protective cover comprising:
   a first end portion;
   a second end portion;
   a lens opening configured to allow access to the lens, the lens opening positioned laterally between the first end portion and the second end portion;
   an outer side;
   an inner side, the inner side comprising a shape that corresponds to a shape of at least the first side of the frame and at least the portion of the lens cleaning system, the inner side further comprising:
      a first cleaning system receiver configured to cover a first portion of the lens cleaning system, the first cleaning system receiver positioned at the first end portion; and
      a second cleaning system receiver configured to cover a second portion of the lens cleaning system, the second cleaning system receiver positioned at the second end portion;
wherein the protective cover is coupled to the frame;
wherein the protective cover is configured to enclose at least the first side and at least the portion of the lens cleaning system; and
wherein the protective cover is configured to reduce a failure of the lens cleaning system due to debris contacting the lens cleaning system.

46. A method of assembling an eyewear system, comprising
providing an eyewear comprising a goggle having a frame and a lens carried by the frame, and a lens cleaning system coupled to the frame and positioned adjacent to the lens; and
releasably attaching a protective cover to eyewear to at least partially cover the frame and the lens cleaning system, the protective cover comprising:
   opposing first and second end portions;
   a lens opening laterally between the first end portion and the second end portion, the lens opening configured to allow access to the lens of the eyewear for visibility through the lens, the lens cleaning system and the lens opening;
   an outer side facing away from the frame;
   an inner side facing toward the frame, the inner side having a contour that corresponds to a shape of at least a first side of the frame and at least a portion of the lens cleaning system, the inner side further comprising:
   a first cleaning system receiver configured to cover a first portion of the lens cleaning system, the first cleaning system receiver positioned at the first end portion; and
   a second cleaning system receiver configured to cover a second portion of the lens cleaning system of the eyewear, the second cleaning system receiver positioned at the second end portion;
   wherein the protective cover is positioned relative to the goggle configured to releasably couple to the frame and movable between an installed position on the frame and a separated position removed from the frame;
wherein the protective cover is positioned on the eyewear in the installed position with the protective cover enclosing at least a first side of the frame and at least the portion of the lens cleaning system, the first side extending across a surface of the frame configured to face away from the wearer when worn; and
wherein the protective cover is positioned to block debris from engaging the lens cleaning system during use of the eyewear system.

47. The eyewear system of claim 24, wherein the lens cleaning system comprises a roll-off system.

### Terminology

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

Conditional language used herein, such as, among others, “can,” “could,” “might,” “may,” “e.g.,” and the like, unless specifically stated otherwise, or otherwise understood within the context as used, is generally intended to convey that certain implementations include, while other implementations do not include, certain features, elements and/or states. Thus, such conditional language is not generally intended to imply that features, elements and/or configurations are in any way required for one or more implementations. The terms “comprising,” “including,” “having,” and the like are synonymous and are used inclusively, in an open-ended fashion, and do not exclude additional elements, features, acts, operations, and so forth. The term “consisting essentially of” can be used anywhere where the terms comprising, including, containing or having are used herein, but consistent essentially of is intended to mean that the claim scope covers or is limited to the specified materials or steps recited and those that do not materially affect the basic and novel characteristic(s) of the claimed invention. Also, the term “consisting of” can be used anywhere where the terms comprising, including, containing or having are used herein, but consistent of excludes any element, step, or ingredient not specified in a given claim where it is used.

Additionally, in the subject description, the word “example” is used to mean serving as an example, instance, or illustration. Any implementations or design described herein as “example” is not necessarily to be construed as preferred or advantageous over other implementations or designs. Rather, use of the word example is intended to present concepts in a concrete manner. Accordingly, all such modifications are intended to be included within the scope of the present inventions. Other substitutions, modifications, changes, and omissions may be made in the design, operating conditions, and arrangement of the preferred and other example implementations without departing from scope of the present disclosure or from the spirit of the appended claims.

From the foregoing, it will be appreciated that specific embodiments of the invention have been described herein for purposes of illustration, but that various modifications may be made without deviating from the scope of the invention. Accordingly, the invention is not limited except as by the appended claims.

We claim:

1. An eyewear system, comprising
a goggle having a frame and a lens carried by the frame;
a lens cleaning system coupled to the frame and positioned adjacent to the lens; and
a protective cover releasably coupled to the goggle and the lens cleaning system, the protective cover comprising:
opposing first and second end portions;
a lens opening laterally between the first end portion and the second end portion, the lens opening configured to allow access to the lens of the eyewear for visibility through the lens, the lens cleaning system and the lens opening;
an outer side facing away from the frame;
an inner side facing toward the frame, the inner side having a contour that corresponds to a shape of at least a first side of the frame and at least a portion of the lens cleaning system, the inner side further comprising:
a first cleaning system receiver configured to cover a first portion of the lens cleaning system, the first cleaning system receiver positioned at the first end portion; and
a second cleaning system receiver configured to cover a second portion of the lens cleaning system of the eyewear, the second cleaning system receiver positioned at the second end portion;
wherein the protective cover is configured to releasably couple to the frame and movable between an installed position on the frame and a separated position removed from the frame;
wherein the protective cover in the installed position encloses at least a first side of the frame and at least the portion of the lens cleaning system, the first side extending across a surface of the frame configured to face away from the wearer when worn; and
wherein the protective cover in the installed position is configured to block debris from engaging the lens cleaning system during use of the eyewear system.

2. The eyewear system of claim 1, wherein the lens cleaning system comprises a roll-off system.

3. The eyewear system of claim 1, wherein the first portion of the lens cleaning system comprises a first canister configured to store at least a first portion of a film for extending across the lens of the eyewear; and the second portion of the lens cleaning system comprises a second canister configured to store at least a second portion of the film.

4. The eyewear system of claim 1, wherein the protective cover has comprises a scraper configured to tear a film of the lens cleaning system.

5. The eyewear system of claim 4, wherein the scraper comprises a first scraper positioned on the first end portion; and a second scraper positioned on the second end portion, the first scraper and the second scraper each defining a rip-off interface configured to facilitate detachably coupling a rip-off film sheet across the lens received of the eyewear.

6. The eyewear system of claim 4, wherein the scraper comprises a plurality of raised blades.

7. The eyewear system of claim 1, wherein the frame of the goggle comprises opposing top and bottom portions, wherein the top portion extends from the first end portion to the second end portion, and the bottom portion extends from the first end portion to the second end portion, and wherein the top portion, the bottom portion, the first end portion, and the second end portion are configured to surround the lens opening of the protective cover.

8. The eyewear system of claim 1, wherein the protective cover is releasably coupled to the goggle by an adhesive, a snap-fit arrangement, or a mechanical fastener.

9. The protective cover of claim 1, further comprising one or more attachment features for securing the protective cover to the goggle, the one or more attachment features comprising a tab comprising a track configured to position the protective cover relative to the eyewear.

10. A protective eyewear system, comprising
an eyewear having a frame, a lens coupled to the frame, and a lens cleaning system coupled to the frame and positioned adjacent to the lens; and
a protective cover coupled to the eyewear, the protective cover comprising:
opposing first and second end portions;
a lens opening laterally between the first end portion and the second end portion, the lens opening configured to allow access to the lens of the eyewear for visibility through the lens, the lens cleaning system and the lens opening;
an outer side facing away from the frame;
an inner side facing toward the frame, the inner side having a shape that corresponds to a shape of at least a first side of the frame and at least a portion of the lens cleaning system, the inner side further comprising:
a first cleaning system receiver configured to cover a first portion of the lens cleaning system, the first cleaning system receiver positioned at the first end portion; and
a second cleaning system receiver configured to cover a second portion of the lens cleaning system of the eyewear, the second cleaning system receiver positioned at the second end portion;
wherein the protective cover is configured to releasably couple to the frame and movable between an installed position on the frame and a separated position removed from the frame;
wherein the protective cover in the installed position encloses at least a first side of the frame and at least the portion of the lens cleaning system, the first side extending across a surface of the frame configured to face away from the wearer when worn; and
wherein the protective cover in the installed position is configured to block debris from engaging the lens cleaning system during use of the eyewear system.

11. The protective eyewear system of claim 10, wherein the lens cleaning system comprises a roll-off system.

12. The protective eyewear system of claim 10, wherein the first portion of the lens cleaning system comprises a first canister configured to store at least a first portion of a film for extending across the lens of the eyewear; and the second portion of the lens cleaning system comprises a second canister configured to store at least a second portion of the film.

13. The protective eyewear system of claim 10, wherein the protective cover has a first scraper positioned on the first end portion; and a second scraper positioned on the second end portion, the first scraper and the second scraper each defining a rip-off interface configured to facilitate detachably coupling a rip-off film sheet across the lens received of the eyewear.

14. The protective eyewear system of claim 10, wherein the frame of the eyewear comprises opposing top and bottom portions, wherein the top portion extends from the first end portion to the second end portion, and the bottom portion extends from the first end portion to the second end portion, and wherein the top portion, the bottom portion, the first end portion, and the second end portion are configured to surround the lens opening of the protective cover.

15. The protective eyewear system of claim 10, wherein the protective cover comprises one or more attachment features for securing the protective cover to the eyewear, the one or more attachment features comprising a tab comprising a track configured to position the protective cover relative to the frame.

16. A protective cover for an eyewear having a lens cleaning system and a frame, the protective cover comprising:
a first end portion;
a second end portion;
a lens opening configured to allow access to a lens of the eyewear, the lens opening positioned laterally between the first end portion and the second end portion;
an outer side;
an inner side, the inner side comprising a shape that corresponds to a shape of at least a first side of the frame of the eyewear and at least a portion of the lens cleaning system, the inner side further comprising:
a first cleaning system receiver configured to cover a first portion of the lens cleaning system of the eyewear, the first cleaning system receiver positioned at the first end portion; and
a second cleaning system receiver configured to cover a second portion of the lens cleaning system of the eyewear, the second cleaning system receiver positioned at the second end portion;
wherein the protective cover is configured to couple to the frame;
wherein the protective cover is configured to enclose at least a first side of the frame and at least the portion of the lens cleaning system, the first side extending across a surface of the frame configured to face away from the wearer when worn; and
wherein the protective cover is configured to reduce a failure of the lens cleaning system due to debris contacting the lens cleaning system.

17. The protective cover of claim 16, wherein the first portion of the lens cleaning system is a first portion of a first canister, the first canister configured to store at least a first portion of a film for cleaning the lens of the eyewear; and
wherein the second portion of the lens cleaning system is a second portion of a second canister, the second canister configured to store at least a second portion of the film.

18. The protective cover of claim 16, further comprising a scraper configured to tear a film of the lens cleaning system.

19. The protective cover of claim 18, wherein the scraper comprises a plurality of raised blades.

20. The protective cover of claim 1, further comprising:
a top portion; and
a bottom portion, opposite the top portion;
wherein the top portion extends from the first end portion to the second end portion;
wherein the bottom portion extends from the first end portion to the second end portion; and
wherein the top portion, the bottom portion, the first end portion, and the second end portion are configured to surround the lens opening of the cover.

21. The protective cover of claim 16, further comprising one or more attachment features for securing the protective cover to the eyewear, the one or more attachment features comprising a tab comprising a track configured to position the protective cover relative to the eyewear.

22. An eyewear system, comprising:
an eyewear comprising:
a frame;
a lens disposed within the frame;
a lens cleaning system for cleaning the lens;
a first side extending across the frame;
a second side opposite the first side, the second side configured to contact a face of a wearer of the eyewear when worn; and
a protective cover removably attachable to the eyewear, the protective cover comprising:
a first end portion;
a second end portion;
a lens opening configured to allow access to the lens, the lens opening positioned laterally between the first end portion and the second end portion;
an outer side;
an inner side, the inner side comprising a shape that corresponds to a shape of at least the first side of the frame and at least the portion of the lens cleaning system, the inner side further comprising:
a first cleaning system receiver configured to cover a first portion of the lens cleaning system, the first cleaning system receiver positioned at the first end portion; and
a second cleaning system receiver configured to cover a second portion of the lens cleaning system, the second cleaning system receiver positioned at the second end portion;
wherein the protective cover is coupled to the frame;
wherein the protective cover is configured to enclose at least the first side and at least the portion of the lens cleaning system; and
wherein the protective cover is configured to reduce a failure of the lens cleaning system due to debris contacting the lens cleaning system.
